(19) United States
(12) Patent Application Publication
(10) Pub. No.: US 2005/0221287 A1
(43) Pub. Date: Oct. 6, 2005

(54) Functional inactivation of cxcr4-mediated responses in growth hormone transgenic mice through socs53 upregulation

(75) Inventors: Carlos Martinez-Alonzo, Madrid (ES); Mario Mellado, Madrid (ES); Jose Rodrigues-Frade, Madrid (ES)

Correspondence Address: CONNOLLY BOVE LODGE & HUTZ LLP, SUITE 800, 1990 M STREET NW, WASHINGTON, DC, 20036-3425, US

(73) Assignee: Consejo Superior de Investigaciones Cientificas Pharmacia Spain, S.A., Alcobendas (Madrid) (ES)

(21) Appl. No.: 10/500,558

(22) PCT Filed: Dec. 31, 2002
(86) PCT No.: PCT/IB02/05789

§ 371 Date: Apr. 4, 2005

Related U.S. Application Data
(60) Provisional application No. 60/343,222, filed on Dec. 31, 2001.

(51) Int. Cl. C12Q 1/70

(52) U.S. Cl. 435/5

(57) ABSTRACT

A method is provided of identifying compounds for use in a pharmaceutical composition having an anti-viral effect against CXCR4-dependent HIV activity comprising: providing a first aliquot of CXCR-4-expressing GH-Tg cells; contacting said first aliquot with HIV particles; providing a second aliquot of CXCR4-expressing GH-Tg cells; contacting said second aliquot with a test ligand; contacting said second aliquot with HIV particles; and isolating virus from said first and said second aliquot of cells, wherein a decrease in the ability to isolate virus from said second aliquot cells indicates said test ligand possess anti-viral activity against HIV. Also provided is a method of identifying compounds for use in a pharmaceutical composition having a therapeutic effect against a disease involving CXCR4-dependent chemotaxis comprising providing a first aliquot CXCR4-expressing GH-Tg cells; contacting said first aliquot with CXCL12; measuring a first migration indes; providing a second aliquot of CXCR4-expressing GH-Tg cells; contacting said second aliquot with CXCL12; contacting said second aliquot with a test ligand; measuring a second migration index; and determing a therapeutic potential. In addition, compounds for us in a pharmaceutical composition having a therapeutic effect against a SOCS3 inhibitable disease are identified by a method comprising: providing a first cell having at least one chemokine receptor expressed thereon, said first cell having been transfected with at least one SOCS construct; contacting said first cell with at least one chemokine, measuring a first migration index; providing a second cell having at least one chemokine receptor expressed thereon, said second cell having been transfected with at least one SOCS construct; contacting said second cell with at least one chemokine; measuring a second migration index; and determining a therapeutic potential subjects having a disease associated with CXCR4-dependent HIV are treated by administering to said subject a therapeutically anti-viral effective amount of a compound that induces the expression of SOCS3 and a pharmaceutically acceptable carrier.

### REFERENCE TO RELATED APPLICATIONS

This Application is a 371 of PCT/IB02/05789, filed Dec. 31, 2002, and claims the priority of U.S. Provisional application 60/343,222, filed Dec. 31, 2001. The entire contents of each priority document is hereby specifically incorporated for all purposes.

### FIELD OF THE INVENTION

The present invention relates generally to intracellular signaling by CXC type chemokine receptors, and particularly, but not by way of limitation, to signaling involving receptor dimers comprising at least one CXCR4. Specifically, the present invention relates to methods to screen for potential therapeutic molecules

### BACKGROUND

The chemokines comprise a large family of low molecular weight (8-10 kDa) cytokines, with chemotactic and pro-activatory effects on different leukocyte lineages (1, 2, 3). Several studies have established the central role of chemokines in a number of physiological situations, including T helper responses, hematopoiesis, angiogenesis, and homeostasis, as well as in pathological conditions such as asthma, tumor rejection, HIV-1 infection, and arteriosclerosis (4-8).

Chemokines mediate their biological effects after binding to specific receptors, members of the seven-transmembrane domain G protein-coupled receptor family (1, 2) Chemokine receptors are promiscuous, as each can bind more than one chemokine; expression is heterogeneous among different cells of the leukocyte lineage and is transcriptionally regulated (3, 5). An exception is the CXCR4 receptor, which binds only CXCL12 (1, 2), a chemokine isolated from stromal cell culture supernatants (9). Its chemotactic properties have been described on peripheral blood lymphocytes (PBL) (10), CD34+ progenitor cells (11), and pre- and pro-B cell lines (12). Knockout (KO) mice lacking the CXCL12 protein (10) and mice lacking the CXCR4 receptor (13, 14) display similar phenotypes: animals die before birth, displaying abnormalities in B cell lymphopoiesis, bone marrow myelopoiesis, lack of blood vessel formation in the gut, severe ventricular septal defects and altered cerebellar neuron migration (13-15).

CXCL12 triggers the association of guanine nucleotide-binding proteins (G proteins) to the CXCR4 (16); the consequence is increased phosphorylation of focal adhesion components, including focal tyrosine kinase (RAFTK/Pyk2), Crk and paxillin (17), CXCL 12 promotes activation not only of signaling molecules that mediate changes in the cytoskeletal apparatus, but also of transcription factors that regulate cell growth, including the p44/42 MAP kinases (Erk and 2) (16), PI3 kinase (17, 19, 20), and NF-,cB (17). The present inventors have recently demonstrated that CXCL12 (14), as well as other chemokines such as CCL2 (MCP-1) and CCL5 (RANTES) (21, 22), exert their effects via dimerization of their receptors and activation of JAK kinases, a protein family originally implicated in cytokine signaling (23). This in turn phosphorylates the chemokine receptors in tyrosine residues and activates the STAT transcription factors (24).

A regulatory feedback mechanism has been identified that shuts down biological responses during cytokine signaling A protein family termed suppressor of cytokine responses (SOCS), whose expression appears to be controlled by the STATs, has a critical role in this regulation (25. 28). The SOCS bind to JAK kinases or to receptors, blocking their activity The regulatory role of SOCS molecules is not limited to the cytokine receptor superfamily, as interaction between the SOCS and the IGF-I receptor has been described, suggesting that SOCS proteins may have a more extensive role in receptor signaling (29).

### SUMMARY OF INVENTION

In characterizing the bGH-Tg mouse phenotype, the present inventors analyzed chemotactic responses to several chemokines and observed impaired in vivo and in vitro responses to CXCL12 Differential RNA display analysis of Tg and non-Tg littennate spleen cells identified a number of candidate genes possibly involved in this impaired response; one of these, SOCS3, pertains to the abovementioned SOCS protein family. Based on the ability of chemokines and cytokines to activate the JAK/STAT pathway, we explored whether SOCS3 also regulates chemokine responses. The present inventors have shown clear evidence that CXCR4 occupancy by CXCL12 upregulates SOCS3, which in turn blocks its signaling pathways, GH upregulation of SOCS3 also regulates CXCL12-mediated responses preventing JAK/STAT pathway activation and Ga; association to the CXCR4 receptor, blocking the chemotactic response. The present invention permits data, derived from bGH-Tg mice in the context of crosstalk between cytokine and chemokine responses, to aid in understanding the functional role of this chemokine/chemokine receptor pair. As the only models available to date were those in which the CXCR4 or CXCL12 deletion is lethal before birth the present invention provides means for relating cytokine-mediated effects to the functional role of CXCR4 inactivation in postnatal life.

According to an aspect of the invention, a method is provided for treating a human having a disease associated with CXCR4-dependent HIV comprising administering to said human a therapeutically anti-viral effective amount of a molecule that induces the expression of SOCS3 and a pharmaceutically acceptable carrier.

According to an aspect of the invention, a method is provided for treating a human having a disease associated with CXCR4-dependent HIV, wherein said molecule binds to GHR.

According to an aspect of the invention, a method is provided to identify compounds for use in a pharmaceutical composition having an anti-viral effect against CXCR4-dependent HIV activity. Aspects of the method comprise: providing a first aliquot of CXCR4-expressing GH-Tg cells; contacting the first aliquot with human immunodeficiency virus (HIV) particles; providing a second aliquot of CXCR4-expressing GH-Tg cells; contacting the second aliquot with a test ligand; contacting the second aliquot with HIV; and isolating virus from the first and the second aliquot of cells, wherein a decrease in the ability to isolate virus from the second aliquot of cells indicates that the test ligand possess anti-viral activity against HIV.

According to an aspect of the invention, a method is provided to identify compounds for use in a pharmaceutical composition having an anti-viral effect against CXCR4-dependent HIV activity, wherein the second aliquot of cells is contacted with a plurality of test ligands.

According to another aspect of the invention, a method is provided to identify compounds for use in a pharmaceutical composition having a therapeutic effect against a disease involving CXCR4-dependent chemotaxis. Aspects of the method comprise: providing a first aliquot of CXCR4-expressing GH-Tg cells; contacting the first aliquot with CXCL12; measuring a first migration index; providing a second aliquot of CXCR4-expressing GH-Tg cells; contacting the second aliquot with CXCL12; contacting the second aliquot with a test ligand; measuring a second migration index; and determining a therapeutic potential.

According to a further aspect of the invention, a therapeutic potential is inversely correlated with the concentration of test ligand required to decrease a migration index. According to a further aspect of the invention, a therapeutic potential may be determined as an ID₅₀, wherein an ID₅₀, is a dose, measured as the concentration of test ligand required to inhibit the migration index by 50%.

According to a still further aspect of the invention, a method is provided to identify compounds for use in a pharmaceutical composition having a therapeutic effect against a disease involving CXCR4-dependent chemotaxis, wherein the disease is associated with aberrant leukocyte recruitment or activation. The disease is selected from the group consisting of arthritis, psoriasis, multiple sclerosis, ulcerative colitis, Crohn's disease, allergy, asthma, AIDS associated encephalitis, AIDS related maculopapular skin eruption, AIDS related interstitial pneumonia, AIDS related enteropathy, AIDS related periportal hepatic inflammation and AIDS related glomerulo nephritis

According to an aspect of the invention, a method is provided to identify compounds for use in a pharmaceutical composition having a therapeutic effect against a disease involving CXCR4-dependent chemotaxis, wherein the cell is a CXCR4 expressing cell selected from the group consisting of thymocytes, CD34+ cells, B lymphocytes, T lymphocytes, dendritic cells, macrophages, neutrophils, and platelets.

According to a further aspect of the invention, a method is provided to identify compounds for use in a pharmaceutical composition having a therapeutic effect against a SOCS3-inhibitable disease comprising: providing a first cell having at least one chemokine receptor expressed thereon, the first cell having been transfected with at least one SOCS construct; contacting the first cell with at least one chemokine; measuring a first migration index; providing a second cell having at least one chemokine receptor expressed thereon, the second cell having been transfected with at least one SOCS construct; contacting the second cell with at least one chemokine; measuring a second migration index; and determining a therapeutic potential.

According to an aspect of the invention, a method is provided to identify compounds for use in a pharmaceutical composition having a therapeutic effect against a SOCS3 inhibitable disease, wherein said cells are HEK cells.

Still other objects and advantages of the present invention will become readily apparent by those skilled in the art from the following detailed description, wherein it is shown and described preferred embodiments of the invention, simply by way of illustration of the best mode contemplated of carrying out the invention. As will be realized the invention is capable of other and different embodiments, and its several details are capable of modifications in various obvious respects, without departing from the invention. Accordingly, the description is to be regarded as illustrative in nature and not as restrictive.

### BRIEF DESCRIPTION OF DRAWINGS

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1. Shows bGH-Tg mice have an impaired chemotactic response to CXCL 12.

FIG. 2. Shows SOCS3 levels are upregulated in bGH-Tg mice.

FIG. 3. Shows IM-9 cells express functional CXCR4 and hGH receptors.

FIG. 4. Shows CXCL12 induces STAT activation in IM-9 cells.

FIG. 5. Shows CXCL12 induces functional STAT translocation to the cell nucleus.

FIG. 6. Shows hGH treatment affects CXCL12-mediated responses.

FIG. 7. Shows SOCS protein expression blocks CXCL12-induced cell responses.

FIG. 8. Shows Lymphoid organ and cerebellum defects in bGH-Tg mice.

FIG. 9. Shows Molecular mechanism of GH influence on CXCL12 signaling.

It is to be noted, however, that the appended drawings illustrate only typical embodiments of this invention and are therefore not to be considered limiting of its scope, for the invention may admit to other equally effective embodiments.

### DETAILED DESCRIPTION OF BEST AND VARIOUS MODES FOR CARRYING OUT THE INVENTION

Reference is made to the figures to illustrate selected embodiments and preferred modes of carrying out the invention. It is to be understood that the invention is not hereby limited to those aspects depicted in the figures.

As used herein, the term “CXCR4” relates to a chemokine receptor. More specifically, the term is used as defined by The International Union of Pharmacology (Murphy et al. International Union of Pharmacology. XXII. Nomenclature for Chemokine Receptors, 52 Pharmacological Reviews 145 (2000), the teachings of which are incorporated by reference for all purposes.

Chemokine receptors are defined by their ability to signal on binding one or more members of the chemokine superfamily of chemotactic cytokines. To date, 18 human proteins have met this definition, and they have been designated CXCR1 through 5, CCR1 through 11, XCR1, and CX3CR1 based on their specific chemokine preferences. Together, chemokine receptors comprise a large branch of the rhodopsin family of cell surface, seven-transmembrane do-main (7TMD), G protein-coupled receptors (GPCRs). In addition, D6 and Duffy (sometimes called the Duffy antigen receptor for chemokines, or DARC) are 7TMD mammalian chemokine-binding proteins that apparently do not signal and therefore are excluded from the systematic nomenclature To date, chemokine receptor-like sequences have been identified in mammals, birds, and fish but not in invertebrates, plants, yeast, or bacteria, suggesting a relatively recent origin. Common features include conserved structure [25-80% amino acid (aa) identity], coupling to the Gi class of G proteins, expression in leukocytes, and chemotactic signaling. The major shared biological function is leukocyte trafficking and dependent processes such as immune surveillance, innate and adaptive immune responses, and various forms of pathological inflammation. Within this general area, however, each chemokine receptor appears to have a specific role, determined by its expression pattern on specific subsets of leukocytes, and by the temporal and spatial specificity of cognate ligand expression. Specific roles have also been delineated in hematopoiesis, angiogenesis, development. Cellular chemokine receptors are exploited as cell entry and disease transmission factors by intracellular pathogens. An example of this are the human immunodeficiency virus (HIV) coreceptor CCR5 in acquired immune deficiency syndrome (AIDS) CXCR4 and other chemokine receptors also function as HIV coreceptors.

Chemokines can be subclassified by structure according to the number and spacing of conserved cysteines into four major groups, given the preferred names CXC, CC, C, and CX3C, which are used in the systematic nomenclatures CXC, CC, and CX3C chemokines all have four conserved cysteines, whereas C chemokines have only two, corresponding to the second and fourth cysteines in the other groups. A small subgroup of CC chemokines has six cysteines. CXC and CX3C chemokines are distinguished by the presence of one (CXC) or three (CX3C) amino acid residues between the first and second cysteines, whereas the first two cysteines of CC chemokines are adjacent. Both the CC and CXC groups have many known members.

The term “subject” as used herein is preferably a mammal, such as a human, but can also be an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

A “therapeutically effective amount” of a compound, as used herein, is an amount which results in the inhibition of one or more processes mediated by the binding of a chemokine to a receptor in a subject with a disease associated with aberrant leukocyte recruitment and/or activation. Examples of such processes include leukocyte migration, integrin activation, transient increases in the concentration of intracellular free calcium and granule release of proinflammatory mediators. Alternatively, a “therapeutically effective amount” of a compound is a quantity sufficient to achieve a desired therapeutic and/or prophylactic effect, such as an amount which results in the prevention of or a decrease in the symptoms associated with a disease associated with aberrant leukocyte recruitment and/or activation.

The amount of compound administered to the individual will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, a therapeutically effective amount of the compound can range from about 0.1 mg per day to about 100 mg per day for an adult. Preferably, the dosage ranges from about 1 mg per day to about 100 mg per day. An antagonist of chemokine receptor function can also be administered in combination with one or more additional therapeutic agents, e.g. theophylline, .β-adrenergic bronchodilators, corticosteroids, antihistamines, antiallergic agents and the like.

The compound can be administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compound can also be administered orally (e.g., dietary), topically, by inhalation (e.g., intrabronchial, intranasal, oral inhalation or intranasal drops), or rectally, depending on the disease or condition to be treated. Oral or parenteral administration are preferred modes of administration.

The compound can be administered to the individual in conjunction with an acceptable pharmaceutical carrier as part of a pharmaceutical composition for treatment of HIV infection, inflammatory disease, or the other diseases discussed above. Formulation of a compound to be administered will vary according to the route of administration selected (e.g., solution, emulsion, capsule). Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

Chemotaxis: CXCR4 dependent migration may be determined, as by way of non-limiting example, as leukocyte chemotaxis assessed on any of eosinophils, peripheral blood mononuclear cells, or HL60 cells, among many cell types. Suitable cell lines may be obtained from many commercial sources, including as non-limiting examples, The American Type Culture Collection (Manassas Va.) or the European collection of Animal Cell Cultures (Porton Downs, Salisbury, U.K.). In an embodiment of the present invention, cells are plated or cultured on Costar Transwell culture inserts (Coming Life Sciences) with a pore size appropriate to the type of cell for which the motility is to be determined. Detailed protocols may be obtained from the manufacturer. The assay medium typically contains CXCR4 and/or the test ligand(s).

According to an aspect of the invention, a method is provided for treating a human having a disease associated with CXCR4-dependent HIV, wherein said molecule binds to GHR.

According to an aspect of the invention, a method is provided to identify compounds for use in a pharmaceutical composition having an anti-viral effect against CXCR4-dependent HIV activity. Aspects of the method comprise: providing a first aliquot of CXCR4-expressing GH-Tg cells; contacting the first aliquot with human immunodeficiency virus (HIV) particles; providing a second aliquot of CXCR4-expressing GH-Tg cells; contacting the second aliquot with a test ligand; contacting the second aliquot with HIV; and isolating virus from the first and the second aliquot of cells, wherein a decrease in the ability to isolate virus from the second aliquot of cells indicates that the test ligand possess anti-viral activity against HIV.

According to an aspect of the invention, a method is provided to identify compounds for use in a pharmaceutical composition having an anti-viral effect against CXCR4-dependent HIV activity, wherein the second aliquot of cells is contacted with a plurality of test ligands.

According to another aspect of the invention, a method is provided to identify compounds for use in a pharmaceutical composition having a therapeutic effect against a disease involving CXCR4-dependent chemotaxis. Aspects of the method comprise: providing a first aliquot of CXCR4-expressing GH-Tg cells; contacting the first aliquot with CXCL12; measuring a first migration index; providing a second aliquot of CXCR4-expressing GH-Tg cells; contacting the second aliquot with CXCL12; contacting the second aliquot with a test ligand; measuring a second migration index; and determining a therapeutic potential.

According to a further aspect of the invention, a therapeutic potential is inversely correlated with the concentration of test ligand required to decrease a migration index. According to a further aspect of the invention, a therapeutic potential may be determined as an ID₅₀, wherein an ID₅₀, is a dose, measured as the concentration of test ligand required to inhibit the migration index by 50%.

According to a still further aspect of the invention, a method is provided to identify compounds for use in a pharmaceutical composition having a therapeutic effect against a disease involving CXCR4-dependent chemotaxis, wherein the disease is associated with aberrant leukocyte recruitment or activation. The disease is selected from the group consisting of arthritis, psoriasis, multiple sclerosis, ulcerative colitis, Crohn's disease, allergy, asthma, AIDS associated encephalitis, AIDS related maculopapular skin eruption, AIDS related interstitial pneumonia, AIDS related enteropathy, AIDS related periportal hepatic inflammation and AIDS related glomerulo nephritis

According to an aspect of the invention, a method is provided to identify compounds for use in a pharmaceutical composition having a therapeutic effect against a disease involving CXCR4-dependent chemotaxis, wherein the cell is a CXCR4 expressing cell selected from the group consisting of thymocytes, CD34+ cells, B lymphocytes, T lymphocytes, dendritic cells, macrophages, neutrophils, and platelets.

According to a further aspect of the invention, a method is provided to identify compounds for use in a pharmaceutical composition having a therapeutic effect against a SOCS3-inhibitable disease comprising: providing a first cell having at least one chemokine receptor expressed thereon, the first cell having been transfected with at least one SOCS construct; contacting the first cell with at least one chemokine; measuring a first migration index; providing a second cell having at least one chemokine receptor expressed thereon, the second cell having been transfected with at least one SOCS construct; contacting the second cell with at least one chemokine; measuring a second migration index; and determining a therapeutic potential.

According to an aspect of the invention, a method is provided to identify compounds for use in a pharmaceutical composition having a therapeutic effect against a SOCS3 inhibitable disease, wherein said cells are HEK cells.

In a preferred embodiment, IM-9 cells, untreated or treated for 16 h with CTx and PTx, (Sigma) are placed (0.25×106 cells in 0. ml) in the upper well of 24-well transmigration chambers (5 gm pore size; Transwell; Costar Corp., Cambridge, Mass.) whose membrane was previously coated with type VI collagen (Sigma; 20 gg/ml), after which 60 nM CXCL12 (PeproTech, Rocky Hill, N.J.) (in 0.6 ml RPMI containing 0.25% BSA) is added to the lower well Plates were incubated (120 min, 37° C.) and cells that migrated to the lower chamber are counted as described (24). When hGH (Genotropin, Pharmacia AB, Uppsala, Sweden) treatment is used, cells (106/ml) are incubated in RPMI 1640 with 0.1% BSA and 10 micrograms/ml recombinant hGH for the time indicated.

When primary mouse cells are evaluated, spleen, lymph node and bone marrow cells are obtained and placed (0.25×106 cells in 0.1 ml) in the upper well of 24-well transmigration chambers (3 gm pore; Transwell) in the conditions described above, In spleen and bone marrow cell preparations, erythrocytes are lysed with NH4Cl (5 min, 37° C.).

Where necessary to deplete freshly isolated primary cells, cells are plated (1×106 cells in 1 ml) in RPMI 1640 supplemented with 0.1% BSA and cultured (2 h, 37° C.). After washing, migration in response to CXCL 12 is evaluated as above.

HEK-293 cell migration was studied in a 96-well microchamber (38) (NeuroProbe Inc., Gaithersburg, Md.). Chemokines at several concentrations were loaded into lower wells (30 pl/well), and cells (200 ˜tl/well, 3×106 cells/ml) in upper wells, Polyvinylpyrrolidone-free filters with 10 gm pores (NeuroProbe) were precoated (2 h, 37° C.) with 20 wg/ml type VI collagen (Sigma). The chamber was incubated (5 h, 37° C.), after which filters were removed and the cells on the upper part wiped off. The cells on the filters were fixed and stained (0.5% crystal violet, 20% methanol). Blue spots developed at positions at which cell migration had occurred, allowing densitometric quantitation of migration (National Institutes of Health Image software). The migration index was calculated by mean spot intensity.

[0CXCR4-Dependent Chemotaxis: A variety of chemokines will induce migration in leucocytes and other cell types. CXCR4 dependent chemotaxis is defined as chemotaxis specifically induced by CXCL12.

Therapeutic Potential: An aspect of the present invention provides means of screening for compounds that bind to receptors that upregulate the expression of SOCS peptides, particularly SOCS3. SOCS3 is capable of inhibiting cellular functions that depend on the binding of CXCL12 to its receptor CXCR4. A non-limiting example of a CXCL12/CXCR4 dependent cellular process is leucocyte chemotaxis. More generally, many cell types migrate, under the influence of the CXCL12/CXCR4 interaction. The present invention concerns ligands that inhibit such cellular processes by binding to a SOCS releasing receptor. Such receptor binding is known in the art to depend on the concentration or dose of the ligand in question. A first definition of Therapeutic Potential is that concentration of test ligand that reduces the migration index by 50%. A second definition of Therapeutic Potential is that dose, termed ID₅₀, of test ligand that reduces the migration index by 50%.

Anti-viral Activity: The ability of chemokines or the derivatives or analogues thereof to bind chemokine receptors and thereby interfere with viral infection or replication can be assayed by various methods known to the art. By way of non-limiting example, DeVico et al. (U.S. Pat. No. 6,214,540) disclose several methods by which anti-viral, and more specifically, anti-HIV activity can be determined. The antiviral activity exhibited by the test ligand may be measured, for example, by easily performed in vitro assays, which can test the compound's ability to inhibit syncytia formation or to inhibit infection by cell-free virus and assess the effects of the compound on cell proliferation and viability. Applying these assays, the relative antiviral activity that a chemokine, derivative and/or analogue exhibits against a given virus or strain of immunodeficiency virus and chemokine, derivative, and/or analogue combination formulation best suited for viral and strain specific inhibitory activity can be determined.

In one embodiment, a cell fusion assay is used to test the ability of the test ligand to inhibit HIV-induced syncytia formation in vitro. Such an assay involves culturing uninfected CD4.sup.+ cells in the presence of chronically HIV-infected cells and the composition containing a chemokine, derivative or analogue to be assayed. For each, a range of concentrations may be tested. This range should include a control culture wherein no chemokine, derivative and/or analogue has been added. Standard conditions for culturing, well known to those of ordinary skill in the art, are used. After incubation for an appropriate period, such as, for example, 24 hours at 37.degree. C., the culture is examined microscopically for the presence of multinucleated giant cells, which are indicative of cell fusion and syncytia formation. Where such assays are performed in the presence and absence of CXCL12 and using cells expressing CXCR4, the antiviral activity of the test ligand against CXCR4 dependent HIV is determined.

### RESULTS

bGH-Tg Mice Have an Impaired Chemotactic Response to CXCL12: the Role of SOCS3 Upregulation

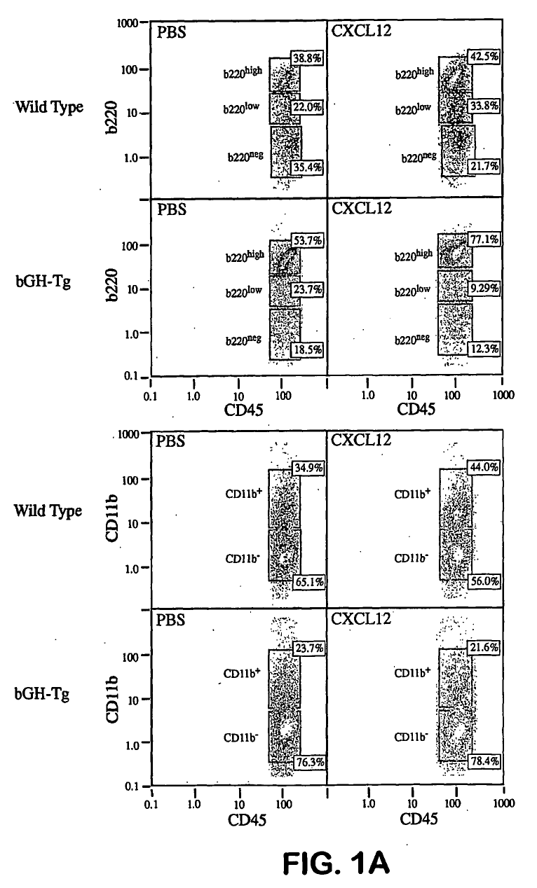

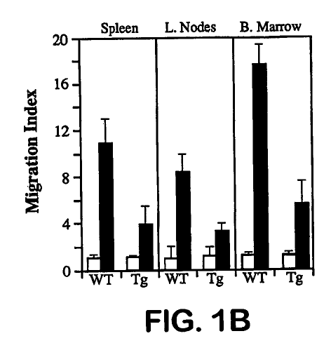

The present inventors have previously shown of both chemokines and cytokines to activate the JAK/STAT pathway. To analyze possible cytolcine/chemolcine crosstalk in signaling, we used a transgenic mouse model expressing a fusion gene coding for bovine growth hormone (bGH-Tg) These mice present renal pathology, diabetes, arthritic and autoimmune disorders, as well as alterations in T cell function. In addition, whereas cell counts indicated only minor variations in T cell subpopulations, bone marrow showed a severe pre-B cell deficit (30, 31). To evaluate the in vivo response to chemokines, CXCL 12 was injected into the peritoneal cavity of bGH-Tg mice and control littermates; after 1 h, cells migrated were recovered and analyzed by flow cytometry using specific cell markers. In CXCL12-injected bGH-Tg-mice, immature B cells and granulocytes/macrophages were absent in the peritoneum, as shown by double staining with b220/CD45 and CD 1 1 b/CD45, respectively (FIG. 1A). No significant alterations were observed in other populations (not shown).

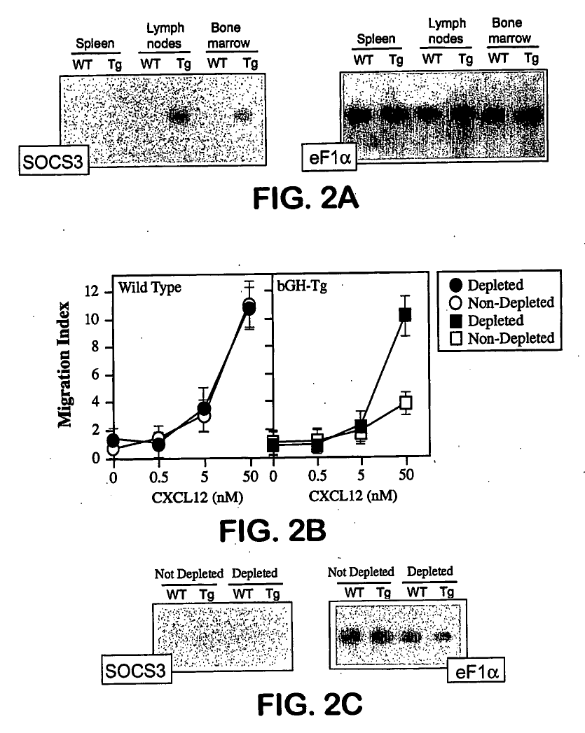

To characterize this impaired in vivo response, spleen, lymph node and bone marrow cells were obtained from three-month-old bGH-Tg mice and allowed to migrate in vitro toward a CXCL12 gradient; CXCL12 migration of cells from non-Tg littermates was measured as a control, Whereas cells from non-Tg mice respond normally to CXCL 12, bGH-Tg mouse cells showed a marked decrease in cell migration (FIG. 1B). Experiments were initiated to analyze the mechanisms underlying the impaired chemotactic responses to CXCL12 in bGH-Tg mice. Differential RNA display analysis by PCR (DDPCR) revealed distinct expression of several genes, among which SOCS3 stood out. DDPCR data were validated by Northern blot; bGH-Tg mouse spleen, lymph node and bone marrow cells express higher SOCS3 levels than cells from non-Tg littermates (FIG. 2A, left). Loading equivalence was confirmed by rehybridizing the membrane with eFla probe (FIG. 2A, right).

The SOCS are proteins implicated in the negative control of cytokine responses, which inhibit the JAK/STAT pathway; their expression is dependent on cytokine receptor signaling. After binding to its receptor, GH activates SOCS3, which regulates GH responses. As the JAK/STAT pathway is involved in CXCL12 signaling, the present inventors tested whether the lack of chemotactic response to CXCL12 is promoted by this GH-mediated upregulation of SOCS3. SOCS3 levels were reduced by in vitro depletion (120 min) of bone marrow cells from bGH-Tg mice in GH-free medium (FIG. 2C), restoring their ability to migrate in response to CXCL12, in contrast to non-depleted cells (FIG. 2B). All together, these experiments assigned a direct role to GH in the negative regulation of CXCL12 signaling and led us to evaluate the role of SOCS proteins in controlling chemotactic responses to this chemokine

CXCL12 Activates the JAKISTAT Pathway:

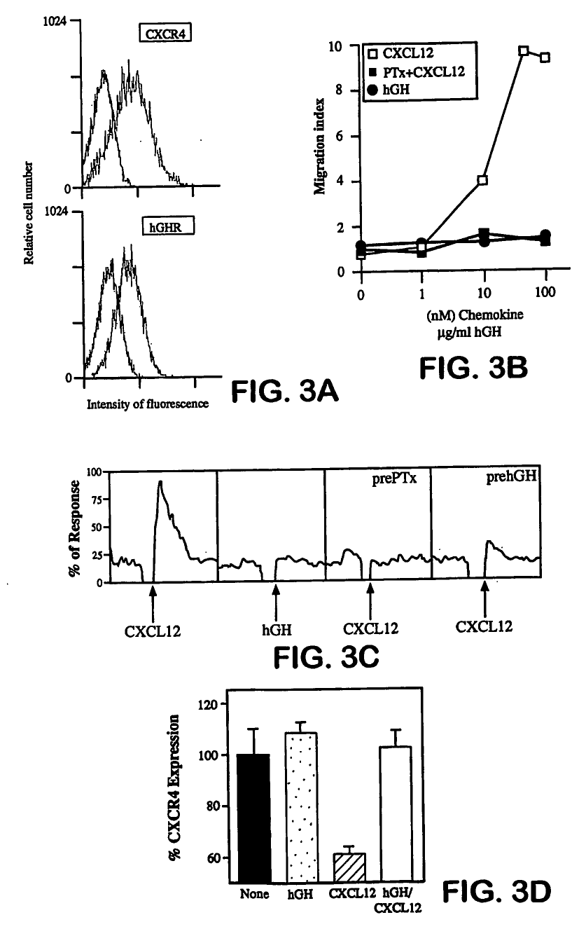

The human IM-9 B cell line expresses the GH receptor as well as CXCR4, shown by flow cytometry analysis using specific antibodies (FIG. 3A). We tested CXCR4-mediated function in these cells by analyzing the IM-9 cell migratory response to increasing CXCL12 concentrations in human type VI collagen-coated transwells. The maximum effect, a five-fold increase in the migration index, is observed at 50 nM CXCL12 (FIG. 3B). As for other cell types (14), this effect was blocked by PTx treatment (FIG. 3B), whereas no effect was seen following incubation with CTx (not shown). As predicted, hGH does not induce cell migration (FIG. 3B), despite its ability to activate these cells (32) and to block the CXCL12 response (FIG. 3C); this effect is not mediated by decreasing surface CXCR4 levels (FIG. 3D). GH pretreatment also prevents CXCL12-mediated CXCR4 internalization, indicating that hGH interferes with CXCL12 signaling, rather than with surface CXCR4 levels

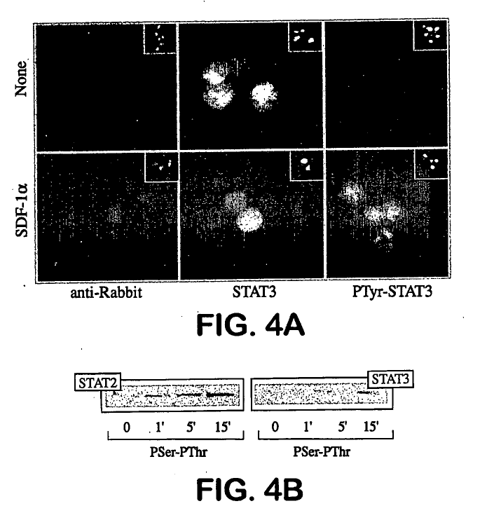

Using the human MOLT4 T cell line, which also expresses the hGHR, the present inventors have shown that CXCR4 undergoes receptor dimerization following CXCL12 binding, as well as rapid association and activation of the JAK/STAT pathway (16). The present inventors observed that signaling through CXCR4 in a B cell line also requires activation of this pathway. In fact, CXCL12 promotes association of JAK1 and JAK3 with CXCR4, followed by their rapid tyrosine phosphorylation. As a consequence, STAT1, 2, 3 and 5, but not STAT4 or 6, are also activated, as shown by their association to the CXCR4 (not shown), as well as their phosphorylation pattern (FIG. 4A).

More interesting is the Ser/Thr phosphorylation of STATs after CXCL12 activation. Using a mixture of phosphoserine-phosphothreonine antibodies, a time course study was performed of STAT3 Ser/Thr phosphorylation, showing correlation with STAT tyrosine phosphorylation kinetics (FIG. 4B). In both cases, maximum phosphorylation occurs 15 min after stimulation.

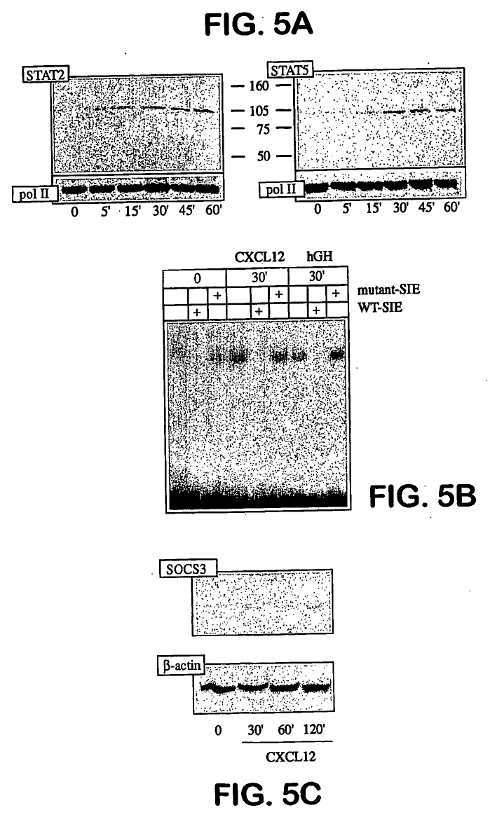

Nuclear extracts of CXCL12-stimulated IM-9 cells were analyzed in Western blot using anti-STAT antibodies, and maximum nuclear translocation was observed 60 min after activation 5A). Nuclear extracts analyzed in Western blot using anti-pol II antibody as a nuclear marker, were free of cytoplasmic contamination, confirmed by developing with an anti-p-actin cytoplasmic marker antibody (not shown). As predicted, SIE binding activity was detected after chemokine treatment, measured by EMSA (FIG. 5B). Protein levels in the nuclear extracts were controlled using a protein detection kit.

CXCL12-Induced SOCS3 Upregulation Inhibits Functional CXCR4 Activity

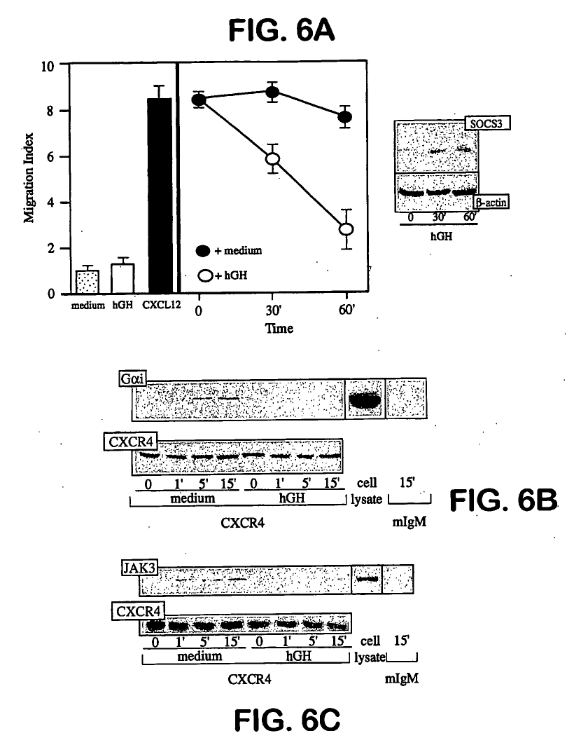

Cytokine activation of the JAK/STAT pathway leads to upregulation of the SOCS, which in turn are implicated in the negative feedback of cytokine signaling. Whether SOCS are upregulated as a consequence of CXCL12-induced STAT translocation in IM-9 cells was evaluated. Only SOCS3 was detected in lysates from CXCL12-stimulated IM-9 cells analyzed in Western blot using antiSOCS antibodies (FIG. 5C), Time-dependent upregulation of SOCS3 was also seen in Western blot analysis of lysates from hGH-treated IM-9 cells using an anti-SOCS3 antibody (FIG. 6A right). GH-mediated SOCS3 upregulation has functional consequences, since the migratory response to CXCL 12 is seriously impaired in GH-pretreated IM-9 cells (FIG. 6A, center).

We next analyzed CXCL12-induced JAK and G: protein association to CXCR4 under conditions in which SOCS3 is upregulated. Serum-starved IM-9 cells, untreated or treated with HGH, were stimulated at different times with 50 nM CXCL12. Cell lysates were immunoprecipitated using anti-CXCR4 antibody and immunoblotted with specific anti-Ga; (FIG. 6B) and anti-JAK3 antibodies (FIG. 6C). Protein loading equivalence was controlled by reblotting membranes with anti-CXCR4 antibody. hGH treatment blocks both JAK3 and Ga; association to the CXCR4. It is therefore concluded that hGH treatment affects CXCL12-mediated responses by directly altering its signaling, without affecting cell surface CXCR4 levels.

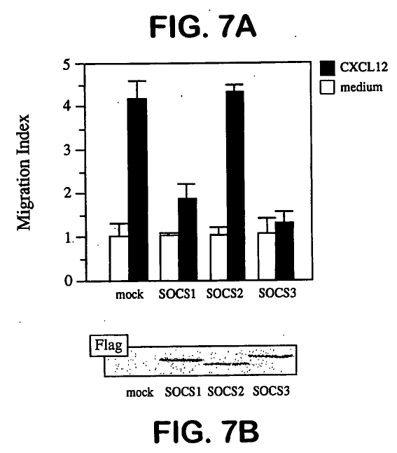

To confirm the role of SOCS3 upregulation in blocking CXCL12 responses, HEK-293 cells transiently transfected with pEF-FLAG-I/mSOCS 1, /mSOCS2 and /mSOCS3 constructs were allowed to migrate in response to a CXCL12 gradient. While no influence was observed on migration of cells expressing SOCS2, a clear reduction was seen in the migration index in SOCS land SOCS3-expressing cells (FIG. 7A). SOCS expression was controlled in each experiment by Western blotting of cell lysates with anti-Flag antibody (FIG. 7B). These data indicate that at least SOCS3, and probably SOCS1, are negative regulators of CXCL12 signaling, and indicate clear crosstalk between chemokine and cytokine signaling,

The bGH-Tg Phenotype is Analogous to That of the CXCR4 Null Mouse

All together, the data presented show that SOCS molecule upregulation by GH affects chemokine responses. Whether the bGH-Tg mouse might be an appropriate model to elucidate the postnatal effect of functional CXCR4-CXCL 12 inactivation was also explored, at least in those cells, organs or tissues in which GHR and CXCR4 are coexpressed. To date, this has not been possible due to the lethal effects at the embryonic stage of CXCR4 or CXCL12 deletion (10, 13, 14).

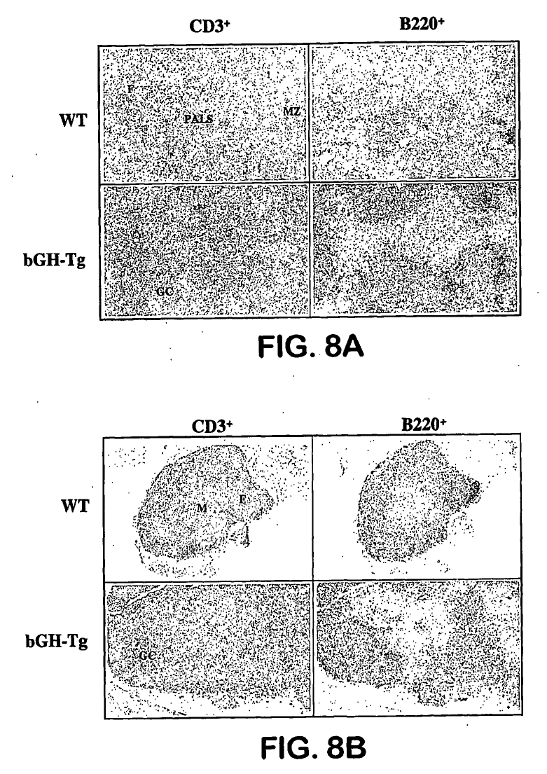

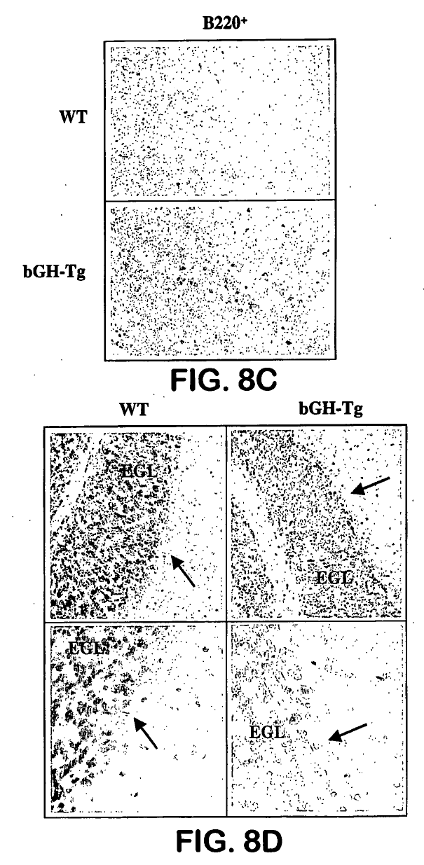

Both CXCR4-′ and CXCL 12−/− mice display identical defects in neuron migration (15), organ vascularization and hematopoiesis (33). In both of these mouse models, T lymphopoiesis is unaffected, whereas B lymphopoiesis is impaired. B cell precursors are greatly reduced in fetal liver and bone marrow, and B cell and granulocyte precursors are released to the periphery (33). To further examine the relationship between bGH-Tg and CXCR4 and CXCL12 knockout mice, we analyzed the structure of lymphoid organs and cerebellum in adult bGH-Tg mice, Immunohistochemistry of the spleen showed alterations of follicular architecture, with disordered B and T cell zones (FIG. 8A); lymph node analysis was characterized by an increase in germinal centers in bGH-Tg mice compared to control littermates (FIG. 8B). Moreover, bGH-Tg mice showed an increase in bone marrow B220+ cells compared to controls (FIG. 8C). These anomalies suggest that bGH overexpression leads to in vivo defects in addition to the inactivation of CXCL12 responsiveness, as irradiated mice reconstituted with CXCR4-′ fetal liver precursors did not exhibit lymphoid organ abnormalities (33). Nonetheless, as in the case of CXCL12-′ mice (13), bGH-Tg mice exhibited cerebellar defects, with diminished density of the peripheral external granule cell layer (EGL) (FIG. 8D, upper) and disorganization of the Purkinje cell layer (FIG. 8D, lower) compared to control littermates. These findings support the analogy between bGH-Tg and CXCL 12-x-nonresponsiveness.

Functional lymphoid microenvironments, organogenesis and leukocyte patrolling are established by cell migration. This requires the integrated action of cell surface ligands and receptors, such as integrins and selectins, with soluble mediators including cytokines, chemokines, and their receptors. Whereas the former group participates in rolling and cell adhesion, the latter plays a key role in the lymphohematopoiesis and cell polarization required for cell motility (34), In addition to their selective role under physiological conditions, chemokines also have an important function in inflammation, wound healing, and angiogenesis (6).

It has been established in recent years that hematopoietic cell growth, differentiation, and function are controlled by the coordinated action of the cytokine-chemokine network (35). Following binding of a cytokine to its receptor on the cell surface, receptor oligomerization takes place, inducing JAK kinase activation. The activated JAK kinases then phosphorylate the cytokine receptors, leading to recruitment and subsequent activation of other signaling molecules, such as the STAT family proteins, among others. Activated STAT proteins translocate to the nucleus and mediate transcription of a range of cytokine-responsive genes (23), including those that code for a family of negative regulators of cytokine signaling, the SOCS proteins, These molecules have recently attracted interest, as they exercise their effect directly on the JAK/STAT pathway. Indeed, the majority of cytokines analyzed to date such as LIF, IL-2, IL,-3, IL-6, GH, IFN-y and leptin, induce several SOCS family members in a tissue-specific manner (36).

Much like cytokines, the chemokines trigger oligomerization of their receptors and activation of the JAK/STAT pathway (37, 38). The similarity among chemokine receptors, including conservation of the DRY motif, nonetheless suggests that oligomerization and JAK/STAT pathway activation are not exclusive to CCR2, in which they were first described (22, 24); CCRS and CXCR4 both activate several JAKlSTAT family members in a cell lineage-dependent fashion. Indeed, dimerization has been described for other GPCR receptors, including the agonist-induced a2-adrenergic (39), opioid (40) and GABA receptors (41); this is also the case of the angiotensin II (42), TSH (43) and a-melanocyte-stimulating hormone (a-MSH) receptors (44), which also activate the JAK/STAT pathway.

CXCR4 is Tyr phosphorylated in response to CXCL 12, as we have shown in the MOLT4 cell line (16), and confirm here in the IM-9 human pre-B cell line. At difference from MOLT4, in IM-9 we observe Tyr phosphorylation and association to CXCR4 of JAK1 and JAK3, but not of JAK2. This indicates a cellular component that bestows specificity on the JAK proteins involved in CXCL12 signaling. CXCL12 also promotes CXCR4 association of several transcriptional STAT proteins, including STAT1, 2, 3, and 5. Tyr phosphorylation of STATs controls the transcriptional activity of this protein family (45), due to its role in STAT dimerization, nuclear translocation, and DNA binding. We also observed CXCL12-mediated Ser/Thr phosphorylation of STATs; the time course of this phosphorylation concurs with STAT dissociation from the Ser/Thr phosphatase PP I, which is critically involved in regulating STAT-mediated gene transcription (46),

As described for cytokines, CXCL12-mediated STAT activation and nuclear translocation promote upregulation of the SOCS3 protein. This CXCL12-mediated SOCS3 upregulation abrogates JAK and G; association to the CXCR4, indicating that SOCS act before STAT activation.

Growth hormone belongs to the cytokine family that also comprises placental lactogen and prolactin; biological effects vary widely, and include skeletal growth during childhood and regulation of a variety of anabolic processes in adult life. Lymphocytes also have receptors for GH, as defined by biochemical, molecular and functional evidence, and GH has been implicated as a growth and differentiation factor in the hematopoietic system (50), After binding GH, the receptor dimerizes and signals through JAK2 kinase. This signaling pathway includes tyrosine phosphorylation of several proteins, among them the latent cytoplasmic transcription factors, STATs. This leads to the upregulation of a variety of genes, among others SOCS2, SOCS3 and CIS mRNA in vivo and in vitro (51). SOCS inhibit receptor signaling to STAT5b via phosphotyrosine-dependent binding interactions with the tyrosine kinase JAK2 (SOCS 1) and/or the cytoplasmic tail of GHR (CIS and SOCS3) (52).

Given the ability of both GH and CXCL12 to stimulate the JAK/STAT pathway, leading to SOCS3 upregulation, the present inventors studied the possible relationship between chemokines and cytokines using CXCL12 and GH as a model system. It has been shown according to the present invention that the IM-9 cells do not migrate in response to hGH, but do so in response to CXCL12. Nonetheless, when cells are pretreated with hGH under conditions that upregulate SOCS3, cell migration in response to a CXCL12 gradient is impaired. When SOCS3 is downregulated, the chemotactic response is recovered. Finally, SOCS3 overexpression in HEK-293 cells prevents a chemotactic response to CXCL12. hGH does not affect cell membrane CXCR4 levels, however, as shown by staining of hGH-treated cells with anti-CXCR4. In vivo experiments have also indicated a possible role of this interaction, since neutrophils purified from patients with acromegaly or hyperprolactinemia show a decrease in in vitro chemotaxis to an N-formylmethionyl-phenylalanine gradient (53). The present inventors used bGH-Tg mice to further study the role of GH in the CXCL12-triggered chemotactic responses. Analysis of bGH-Tg mice also shows upregulated SOCS3 levels and several anomalies, including renal pathology, diabetes, hypertension, sterility, neuropathies, autoimmune disorder (30), significant alterations in T cell function, and loss of pre-B cells in bone marrow (31), indicating a direct GH effect in immune response regulation, directly or mediated by its influence on chemokine responses. These mice have severely reduced B cell lymphopoiesis, reduced myelopoiesis in fetal liver, and virtual absence of myelopoiesis in bone marrow, while T cell lymphopoiesis is unaffected (31).

CXCR4 and CXCL12 deficiency is lethal at embryonic stages, with defects in the developing hematopoietic system (33). The present inventors studied the role of CXCL12 in bGH-Tg mice, a model that is preferentially affected postnatally In vitro analysis of the chemotactic response to CXCL12 reveals that it is impaired in spleen, lymph node and bone marrow cells of bGH-Tg mice as compared to non-Tg controls. Migration was restored to normal levels when cells from bGH-Tg animals were GH-depleted by in vitro starvation. When in vivo CXCL12-mediated responses were analyzed following CXCL12 injection into the peritoneal cavity, a marked reduction in migrating cells was observed in bGH-Tg mice compared to non-Tg littermates. These results assign a potential role to GH in CXCL12-mediated responses, in accordance with the upregulation of SOCS3 mRNA levels in cells from these mice.

Histological analyses of bGH-Tg mice resemble those of CXCR4- and CXCL12-deficient mice, as bGH overexpression leads to EGL and Purkinje cell layer abnormalities, and deletion of CXCR4 or CXCL12 also leads to defects in these two cerebellar cell layers 5). Mice reconstituted with CXCR4-deficient fetal liver cells show well-formed lymph node and spleen architecture, suggesting that CXCL12 responsiveness does not affect lymphoid organ formation.

Conversely, bGH-Tg mice had abnormalities in lymph node and spleen structure This discrepancy may be due to the possibility that GH affects not only the CXCL12-mediated response, but also that of other chemokines or cytokines whose receptor is present on GHR-expressing cells. Indeed, SOCS upregulation has been described in LIF, IL-2, IL-3, IL-6. IFN-y and leptin signaling (36); responses to these cytokines may thus be affected in bGH-Tg mice and contribute to their phenotype

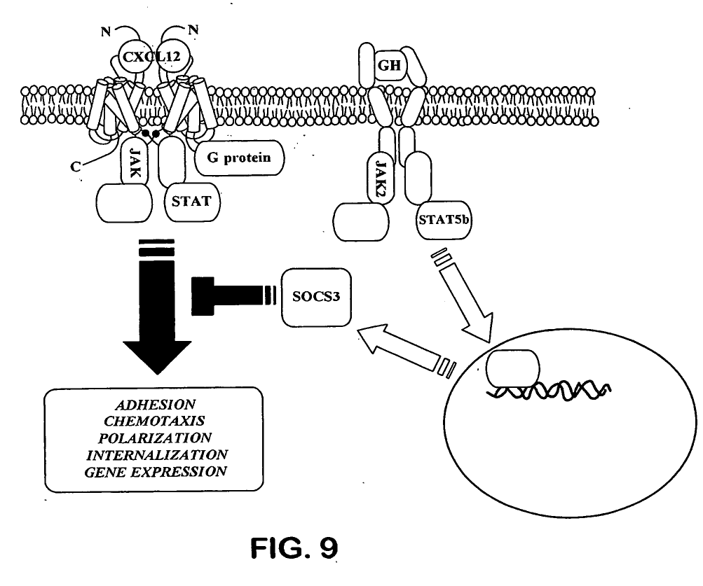

Overall, identification of the chemokine-activated JAK/STAT/SOCS pathway has opened a new avenue in signal transduction research, and has integrated this pathway with those of cytokine signaling (FIG. 9). Cytokine transgenic mice are used conditional models to analyze chemokine-and chemokine receptor-deficient phenotypes, as the defect appears only in cells co-expressing the chemokine receptor and the corresponding cytokine receptor. It is thus critical to identify the cytokine % hemokine combination needed by each lineage during development, as well as those required by cells mobilized during normal immune responses and the inflammatory response. These types of studies form the basis for developing screens for specific pharmacological inhibitors of cytokine and chemokine signaling to interfere with inflammatory responses, as well as other process, such as HIV infection.

### Experiemental Procedures

Biological Materials

IM-9 cells (ATCC CCL159) and HEK-293 cells (ATCC TIB202) were from the American Type Culture Collection (ATCC, Manassas, Va.). Antibodies used include anti-CXCB4 and. anti-hGH receptor mAb generated in our laboratory (16, 32), horseradish peroxidase (PO)-labeled anti-PTyr mAb (4G10) (Upstate Biotechnology, Lake Placid, N.Y.), anti-phosphothreonine and anti-phosphoserine mAb (Calbiochem, San Diego, Calif.), anti-β₂-microglobulin (Pharmingen, San Diego, Calif.), FITC-labeled anti-CD3 (Southern Biotechnologies, Birmingham, Ala.), FITC-anti-CD11b and PE-b220 (Pharmingen); rabbit anti-PTyr (Promega, Madison, Wis.); anti-Gα₁, anti-Gαₛ, anti-STAT2 and anti-STAT3, anti-SOCS3, anti-pol II (Santa Cruz, Santa Cruz, Calif.), and anti-β-actin (Sigma, St. Louis, Mo.). For immunostaining, anti-STAT3 and anti-phospho-STAT3 antibody (New England Biolabs, Beverly, Mass.) were used.

Flow Cytometry Analysis

Cells were centrifuged (250×g, 10 min, room temperature), plated in V-bottom 96-well plates (2.5×105 cells/well) and incubated with a biotin-labeled mAb (1 gg/50 R1/well, 60 min, 4° C.). Cells were washed twice in PBS with 2% bovine serum albumin (BSA) and 2% FCS and centrifuged (250×g, 5 min, 4° C.). Fluorescein isothiocyanate-labeled streptavidin (Southern Biotechnologies) was added, incubated (30 min, 4° C.) and plates washed twice. Cell-bound fluorescence was determined in a Profile XL flow cytometer at 525 run (Coulter, Miami, Fla.).

Immunocytochemistry and Histological Analysis

IM-9 cells were cultured in RPMI with 1% BSA for 2 h, alone or stimulated with 50 nM “CXCL12. Cells were washed with PBS at room temperature (RT), fixed with 100% methanol (10 min, −20° C.) and permeabilized with 0.2% Triton X 100 in TBS (15 min, RT). Cells were washed three times with TBS (5 min, RT) and non-specific binding sites blocked with 5% normal goat serum in TBS (45 min, RT). After washing with TBS, cells were incubated with anti-STAT3 or anti-phospho-STAT3 mAb diluted in TBS, 2% BSA, 2% normal goat serum, followed by Cy3-labeled anti-rabbit IgG antibody (Jackson Immunoresearch, West Grove, Pa.). Vectashield mounting medium with DAPI (Vector Laboratories, Burlingame, Calif.) was added and cells analyzed in a Leica fluorescence microscope, For histological analysis, organ sections (4 gm) fixed in 5% formalin and embedded in paraffin were hematoxylin-eosin stained. For immunostaining, tissue sections were deparaffinized and treated with 3% HO, in 60% methanol to block endogenous peroxidase activity, followed by microwave treatment in 10 mM sodium citrate buffer and incubation with 10% normal mouse serum to avoid non-specific binding. Sections were then incubated with a biotin blocking kit (Dako), stained with a biotin-conjugated anti-B220 antibody (Southern Biotechnology, Birmingham, Ala.), and developed with the StrepABComplex/HRP kit (Dako, Glostrup, Denmark), Peroxidase activity was detected using FAST DAB (Sigma). CD3 was detected using a rabbit antihuman CD3 antibody, followed by a peroxidase-conjugated goat anti-rabbit antibody (both from Dako),

Cell Migration

IM-9 cells, untreated or treated for 16 h with CTx and PTx, (Sigma) were placed (0.25×106 cells in 0. ml) in the upper well of 24-well transmigration chambers (5 gm pore size; Transwell; Costar Corp., Cambridge, Mass.) whose membrane was previously coated with type VI collagen (Sigma; 20 gg/ml), after which 60 nM CXCL12 (PeproTech, Rocky Hill, N.J.) (in 0.6 ml RPMI containing 0.25% BSA) was added to the lower well Plates were incubated (120 min, 37° C.) and cells that migrated to the lower chamber were counted as described (24). When hGH (Genotropin, Pharmacia AB, Uppsala, Sweden) treatment was used, cells (106/ml) were incubated in RPMI 1640 with 0.1% BSA and 10 gg/ml recombinant hGH for the time indicated.

When primary mouse cells were evaluated, spleen, lymph node and bone marrow cells were obtained and placed (0.25×106 cells in 0.1 ml) in the upper well of 24-well transmigration chambers (3 gm pore; Transwell) in the conditions described above, In spleen and bone marrow cell preparations, erythrocytes were lysed with NH4Cl (5 min, 37° C.).

In those experiments in which depletion of fresh isolated primary cells was necessary, cells were plated (1×106 cells in 1 ml) in RPMI 1640 supplemented with 0.1% BSA and cultured (2 h, 37° C.). After washing, migration in response to CXCL 12 was evaluated as above.

HEK-293 cell migration was studied in a 96-well microchamber (38) (NeuroProbe Inc., Gaithersburg, Md.). Chemokines at several concentrations were loaded into lower wells (30 pl/well), and cells (200 ˜tl/well, 3×106 cells/ml) in upper wells, Polyvinylpyrrolidone-free filters with 10 gm pores (NeuroProbe) were precoated (2 h, 37° C.) with 20 wg/ml type VI collagen (Sigma). The chamber was incubated (5 h, 37° C.), after which filters were removed and the cells on the upper part wiped off. The cells on the filters were fixed and stained (0.5% crystal violet, 20% methanol). Blue spots developed at positions at which cell migration had occurred, allowing densitometric quantitation of migration (National Institutes of Health Image software). The migration index was calculated by mean spot intensity.

In Vivo Biological Activity of CXCL12

Mice (3-month-old bGH-Tg and non-Tg littermates) were injected intraperitoneally with 1 gg CXCL12 in 400 gl sterile PBS or PBS alone. Mice were sacrificed 6 h after injection and migrated cells extracted from the peritoneal cavity injecting 6 ml of sterile PBS Cells were counted, centrifuged (250×g, 10 min), and the distinct cell populations enumerated by flow cytometry analysis using specific cell surface markers.

Immunoprecipitation, SDS-PAGE and Western Blot Analysis

CXCL12-stimulated cells (10×106) were lysed in a detergent buffer (20 mM triethanolamine pH 8.0, 300 mM NaCl, 2 mM EDTA, 20% glycerol, 1% digitonin, with 10 gM sodium orthovanadate, 10 gg/ml leupeptin and 10 gg/ml aprotinin) for 30 min at 4° C. with continuous rocking, then centrifuged (15,000×g, 15 min). Immunoprecipitation was performed essentially as described (24). Immunoprecipitates or protein extracts were separated in sodium dodecyl sulphate polyacrylamide gel electrophoresis (SDS-PAGE) and transferred to nitrocellulose membranes. Western blot analysis was as described, using 2% BSA in TBS as blocking agent for anti-phosphotyrosine analyses. When stripping was required, membranes were incubated (60 min, 60° C.) with 62.5 mM Tris-HCl pH 7.8, containing 2% SDS and 0.5% (3-mercaptoethanol. In all cases, protein loading was controlled using a protein detection kit (Pierce) and, when necessary, by reprobing the membrane with the immunoprecipitating antibody.

Preparation of Nuclear Extracts

Nuclear extracts were prepared from CXCL12-stimulated IM-9 cells (10×106) (23). Briefly, cells were washed with ice-cold PBS, resuspended in 1 ml of buffer A (50 MM NaCl, 0.5 M sucrose), incubated (2 min, 4° C.) and pelleted (4500×g, 3 min, 4° C.) They were then resuspended in 1 ml of buffer B (50 mM NaCl, 25% glycerol), pelleted (4500×g, 3 min, 4° C.); and incubated in 60 wl of buffer C (350 mM NaCl, 25% glycerol) for 30 min at 4° C. with continuous rocking. After centrifugation (20,000×g, 20 min, 4° C.), supernatants containing nuclear extracts were aliquoted and stored at −80° C. All buffers contained 0.5 mM spermidine, 0.15 mM spermine, 0.1 mM EDTA, 10 mM HEPES pH 8, 0.5 mM PMSF, 2 gg/ml leupeptin, 3 gg/ml pepstatin, 0.2 IU/ml aprotinin, 1.75 mM P-mercaptoethanol, 1 mM orthovanadate and 10 mM NaF. For Western blot analysis, 20 gg of each nuclear extract were separated in SDS-PAGE and transferred to nitrocellulose membranes. Western blot analysis was as above, and was developed using antiSTAT3 and -STAT5b antibodies.

Electrophoretic Mobility Shift Assay (EMSA)

Nuclear extracts (10 gg), prepared as above from untreated or chemokine-treated cells, were analyzed in binding reactions. Extracts were incubated with 0.5 ng 32P-end-labeled double-stranded oligodeoxynucleotides containing the sis-inducible element (SIE) of the c-fos promoter sequence [GGGGTGCATTTCCCGTAAATCTTGTCT] SEQ ID No. 1 (wild type; wt-SIE); where indicated, a mutant version was used that is unable to bind STAT proteins [GGGGTGCATCCA CCGTAAATCTTGTCT] SEQ ID No. 2 (mut-SIE). The binding reaction was carried out in EMSA buffer for 30 min at room temperature (final volume 10 microliters), with 1.5 microgram of poly(dI-dC) and, where indicated, a 20-fold molar excess of unlabeled SIE or nonspecific oligonucleotide competitor. Protein-DNA complexes were resolved on a 4.5% polyacrylamide gel using 0.5× Tris-borate-EDTA running buffer. EMSA buffer contained 10 mM Tris pH 7.5, 50 mM NaCl, 1 mM dithiothreitol (DTT), mM EDTA and 5% glycerol. Protein amount in nuclear extracts was confirmed before Western blot or EMSA using a protein detection kit (Pierce).

Cell Transfection.

Human embryonic kidney cells (HEK-293) were transiently transfected with pEF-FLAG-I/mSOCS 1, mSOCS2 or mSOCS3 constructs (kindly donated by Dr. T. Willson, Walter and Eliza Hall Institute of Medical Research, Victoria, Australia) with lipofectamine (Gibco-BRL Gaithersburg, Md.) following manufacturer's protocols.

Northern Hybridization

Total RNA from 3-month-old bGH Tg and non-Tg littermate mouse tissues and IM-9 cells untreated or treated (60 min, 37° C.) with 60 nM CXCL12 or 10 gg/ml hGH, was extracted using Tri-reagent (Sigma). RNA samples were electrophoresed on denaturing formaldehyde-agarose gels and transferred to a nylon membrane (Hybond N+, Amersham Pharmacia Biotech AB, Uppsala, Sweden). Membranes were hybridized with 3zP-labeled cDNA from the pEF-FLAG-I/mSOCS3 construct.

### Figure Legends

FIG. 1. bGH-Tg mice have an impaired chemotactic response to CXCL 12. A) CXCL12 (1 mM) was injected into the peritoneum of bGH-Tg mice and of control littermates. After 60 min, cells migrated into the peritoneal cavity were recovered and the cell populations characterized by flow cytometry using specific cell markers. The figure shows specific staining of b220 and CD1 lb cells in the lymphocyte population, as shown by CD45 staining; the percentage of each population is also indicated. The results are shown of one representative experiment of five performed. B) CXCL12 (50 nM)-mediated cell migration of spleen, lymph node and bone marrow cells from bGH-Tg mice or wild type littermates were evaluated in 24-well transmigration chambers. Cells suspended in RPMI 1640 with 1% BSA were allowed to migrate (2 h, 37° C., 5% COZ). Cells that migrated to the lower chamber were counted and expressed as a migration index, calculated as the x-fold increase in migration observed over the negative control (PBS) Data represent the mean of triplicate determinations, with SD indicated.

FIG. 2. SOCS3 levels are upregulated in bGH-Tg mice, A) Northern analysis of SOCS3 mRNA from spleen, lymph nodes and bone marrow of bGH-Tg mice compared to control littermates (left). As a control, the membrane was rehybridized using eFla as a probe (right), B) Bone marrow cells from bGH-Tg mice and control littermates were GH-depleted in vitro (2 h 37° C.) and allowed to migrated in response to CXCL12 (0.5-50 nM). As a control, cell migration in response to CXCL12 was evaluated in freshly isolated (non-depleted) cells from the same mice. The migration index was calculated as in FIG. 113. C) Northern analysis of SOCS3 mRNA from the bone marrow cells described in B (left), As a loading control, the membrane was rehybridized using eF 1 a as probe (right).

FIG. 3. IM-9 cells express functional CXCR4 and hGH receptors. A) Cells as indicated were incubated with biotin-labeled CXCR4-O1 or hGHR-05 mAb, followed by FTTC-streptavidin. The figure shows mAb binding compared with that of a negative control (mIgM), B) hGH (1-100 gg/ml) and CXCL12 (1-100 nM)-induced chemotaxis of IM-9 cells, untreated or pretreated with PTx (0.4 gg/ml, 16 h, 37° C., 5% CO₂ were assayed in chemotaxis chambers as in FIG. 1B. C). CXCL12 (50 nM)- or hGH (10 wg/ml)-induced Ca z+ mobilization in IM-9 cells, untreated or preincubated with PTx (0.4 gg/ml, 16 h, 37° C.) or hGH (10 gg/ml, 60 min. 37° C.), was determined at 525 nm in a flow cytometer. D) Surface CXCR4 expression levels were determined in IM-9 cells treated with hGH (10 gg/ml; 60 min, 37° C.j, with CXCL12 (50 nM; 30 min, 37° C.), or with hGH (10 gg/ml; 60 min, 37° C.) followed by CXCL12 (50 nM; 30 min, 37° C.). Levels were determined by flow cytometry as in FIG. 3A and are expressed as a percentage of the CXCR4 receptor in untreated cells (none), with SD indicated.

FIG. 4. CXCL12 induces STAT activation in IM-9 cells. A) Immunohistochemical analysis of IM-9 cells alone or treated with CXCL12 (50 nM; 15 min, 37° C.) using anti-STAT3 and anti-Ptyr-STAT3 antibody. Nuclear staining is shown (insert, upper right). B) STAT2 and -3 serine/threonine phosphorylation was determined in lysates of CXCL12 (50 nM)-activated IM-9 cells immunoprecipitated with anti-PSer/PThr and assayed in western blot with anti-STAT2 and anti-3 antibodies

FIG. 5. CXCL12 induces functional STAT translocation to the cell nucleus. A) IM-9 cells were stimulated with CXCL12 (50 nM) at the times indicated and nuclear extracts analyzed in western blot using anti-STAT2 and -5 antibodies. Equivalent protein loading was controlled by developing the membrane with anti-pol II antibodies. B) Nuclear extracts from IM-9 cells alone, or treated with CXCL12 (50 nM) or hGH (10 gg/ml) for 30 min at 37° C., were incubated with 32Pend-labeled wt-SIE (0.5 ng) in EMSA buffer. Where indicated, a 20-fold molar excess of unlabeled wt-SIE or mut-SIE oligonucleotide was added. C) Lysates from CXCL12 (50 nM)-activated IM-9 cells were immunoblotted with anti-SOCS3 antibody (upper) Protein loading was controlled by reprobing the membrane with anti-P actin mAb (lower).

FIG. 6. hGH treatment affects CXCL12-mediated responses. A; Untreated or hGH (10 gg/ml)-treated cells (60 min, 37° C.) were allowed to migrate in response to CXCL12 (50 nM) (left); the migration index was calculated as in FIG. 1B. Effect of hGH (10 gg/ml, 37° C.) pretreatment on CXCL12-induced IM-9 cell migration (center); the migration index was calculated as before. Presence of SOCS3 in lysates from cells used in the migration assay was evaluated by western blot analysis (right, upper). Protein loading was controlled by reprobing the membrane with an anti-p actin mAb (right, lower), B) Lysates from CXCL12 (10 nM)-activated IM-9 cells that were untreated or hGH (10 wg/ml)-treated, were immunoprecipitated with CXCR4-O1 or control mIgM antibodies and the western blot developed with anti-Ga; antibody (upper). As a positive control, unprecipitated IM-9 cell lysates were tested with anti-Ga; antibody. To control for protein loading equivalence, the blot was reprobed with the immunoprecipitating anti-CXCR4 antibody (lower) C) Cell lysates treated and immunoprecipitated as in B were immunoblotted with anti-JAK3 antibody (upper), As a positive control, unprecipitated IM-9 cell lysates were tested with antiJAK3 antibody. Protein loading equivalence was controlled as in B (lower).

FIG. 7. SOCS protein expression blocks CXCL12-induced cell responses. A) PEF-FLAG-I/mSOCS 1-, mSOCS2-, and mSOCS3-transfected HEK-293 cells or mock-transfected controls were allowed to migrate in response to 10 nM CXCL12 for 5 h at 37° C. (Materials and Methods). The migration index was calculated by mean spot intensity with respect to background (unstimulated cell migration under each condition). B) SOCS expression in migrating cells was confirmed by immunoblot of transfected cells with anti-FLAG antibody Protein loading was controlled using a protein detection kit (Pierce).

FIG. 8. Lymphoid organ and cerebellum defects in bGH-Tg mice. A) Immunostaining of bGH-Tg and control mouse spleen sections with anti-CD3 and anti-B220 antibodies shows altered follicular structure in the presence of the bGH transgene. Original magnification 10×, F: follicle, MZ: marginal zone, PALS: peri-arteriolar lymphoid sheath B) Inguinal lymph node staining with the same antibodies shows that bGH overexpression enhances the presence of germinal centers (arrows). Original magnification 10×, F: follicle, M: medulla, GC: germinal center. C) Expression of the bGH transgene results in an increase in B220′ cell numbers in bone marrow, Original magnification 20×. D) Hematoxylin-eosin staining of cerebellum shows defects in bGHTg animals. Upper panels show the decreased overall cell density in EGL compared to control littermates. The figure also shows disorganization of the Purkinje cell layer bordering the EGL (arrows). Original magnification 20× Lower panels show a detail of Purkinje cell layer disorganization. Original magnification 40×.

FIG. 9. Molecular mechanism of GH influence on CXCL12 signaling. GH binding promotes receptor dimerization, which in turn activates the JAK2/STAT5b pathway; this signaling pathway is shared with CXCL12, a chemokine that binds the CXCR4. The nuclear translocation of STAT5b induces upregulation of SOCS3 proteins, which block CXCL12-promoted responses via the CXCR4. These interactions provide a link between GH signaling and G protein-coupled chemoattraction through SOCS upregulation.

The foregoing description of the invention illustrates and describes the present invention. Additionally, the disclosure shows and describes only the preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

### BIBLIOGRAPHY

1. Baggiolini, M., Dewald, B. and Moser, B. (1997). Human chemokines: an update. Ann. Rev, Immunol. 15, 675-705.
2. Rollins, B. J. (1997). Chemokines. Blood 90, 909-928.
3. Mackay, C. R., Chemokine: immunology's high impact factors, 2 Nat. Immunol, 95 (2001).
4. Schall T J, Bacon K B. Chemokines, leukocyte trafficking and inflammation, Curr Opin. Immunol., 865 (1994).
5. Rossi D, Zlotnik A. (2000). The Biology of Chemokines and their Receptors. Annu. Immunol. 18, 217-42.
6. Gerard, C. and Rollins, B. J. (2001). Chemokines and diseases. Nat. Immunol. 2, 108-115
7. Moser, B. and Loetscher, P. (2001). Lymphocyte traffic control by chemokines. Nat. Immunol. 2, 123-128.
8. Luther, S. A. and Cyster, J. G. (2001). Chemokines as regulators of T cell differentiation. Nat, Immunol. 2, 102-107.
9. Bleul, C. C., Fuhlbrigge, R. C., Casasnovas, J. M., Aiuti, A. and Springer, T. A. (1996). A highly efficacious lymphocyte chemoattractant, stromal cell-derived factor SDF-1 J. Exp. Med. 184, 101-1109.
10. Nagasawa, T., Hirota, S., Tachibana, K., Takakura, N., Nishikawa, S., Kitamura, Y, Yoshida, N., Kikutani, H. and Kishimoto, T. (1996). Defects of B cell lymphopoiesis and bone marrow myelopoiesis in mice lacking the CXC chemokine PBSF/SDF—Nature 382, 635-638.
11. Aiuti, A., Webb, I. J., Bleul, C., Springer, T. A. and Gutierrez-Ramos, J. C. (1997). The chemokine SDF-1 is a chemoattractant for human CD34+ hematopoietic progenitor cells and provides a new mechanism to explain the mobilization of CD34′ progenitors to peripheral blood. 185 J. Exp. Med, 11 (1997).
12. D'Apuzzo, M., Rolink, A., Loetscher, M., Hoxie, J. A., Clark-Lewis, I., Melchers, F., Baggiolini, M. and Moser, B. (1997). The chemokine SDF-1, stromal cell-derived factor 1, attracts early stage B cell precursors via the chemokine receptor CXCR4, Eur. J. Immunol. 27, 1788.
13. Zou, Y-R., Kottmann, A. H., Kuroda, M., Taniuchi, I. and Littman, D. R. (1998). Function of the chemokine receptor CXCR4 in haematopoiesis and in cerebellar development. Nature 393, 595-599.
14. Tachibana, K., Hirota, S., Iizasa, H., Yoshida, H., Kawabata, K., Kataoka, Y., Kitamura, Y., Matsushima, K., Yoshida, N., Nishikawa, S., Kishimoto, T. and Nagasawa, T. (1998). The chemokine receptor CXCR4 is essential for vascularization of the gastrointestinal tract, Nature 393, 591-594.
15. Ma, Q., Jones, D., Borghesani, P. R., Segal, R A., Nagasawa, T., Kishimoto, T., Bronson, R T. and Springer, T A. (1998). Impaired B-lymphopoiesis, myelopoiesis, and derailed cerebellar neuron migration in CXCR4- and SDF-1-deficient mice. Proc. Natl. Acad. Sci. USA 95, 9448-9453.
16. Vila-Coro, A. J., Rodrfguez-Frade, J. M., Martfn de Ana, A. M., Moreno-Ortfz, M. C., Martfnez-A., C. and Mellado, M. (1999). The chemokine SDF-1a triggers CXCR4 receptor dimerization and activates the JAK/STAT pathway. FASEB J. 13, 1699-1710.
17. Wang, J. F., Park, I. W, and Groopman, J. E. (2000). Stromal cell-derived factor-1alpha stimulates tyrosine phosphorylation of multiple focal adhesion proteins and induces migration of hematopoietic progenitor cells: roles of phosphoinositide-3 kinase and protein kinase C. Blood 95, 2505-2513.
18. Dutt, P., Wang, J. F. and Groopman, J. E. (1998). Stromal cell-derived factor-1 alpha and stem cell factor/kit ligand share signaling pathways in hemopoietic progenitors: a potential mechanism for cooperative induction of chemotaxis. J. Immunol. 161, 3652-3658.
19. Ganju, R. K., Brubaker, S. A., Meyer, J., Dutt, P., Yang, Y., Qin, S., Newman, W. and Groopman, J. E, (1998). The a-chemokine, stromal cell-derived factor-1a, binds to the transmembrane G-protein-coupled CXCR4 receptor and activates multiple signal transduction pathways. J. Biol. Chem. 273, 23169-23175.
20. Vicente-Manzanares, M., Rey, M., Jones, D. R., Sancho, D., Mellado, M., Rodriguez Frade, J. M., del Pozo, M. A., Yanez-M6, M., Martin de Ana, A., Martfnez-A., C., Merida, I. and Sanchez-Madrid, F. (1999). Involvement of phosphatidylinositol 3-kinase in stromal cell-derived factor-1a-induced lymphocyte polarization and chemotaxis. J. Immunol. 163, 4001-4012.
21. Rodrfguez-Frade, J. M., Vila-Coro, A. J., Martfn de Ana, A. M., Nieto, M., Sanchez-Madrid, F., Proudfoot, A. E. I., Wells, T. N. C., Martfnez-A., C. and Mellado, M. (1999). Similarities and differences in RANTES- and (AOP)-RANTES-triggered signals: implications for chemotaxis. J. Cell Biol. 144, 755-765.
22. Rodriguez-Frade, J. M., Vila-Coro, A., Martfn de Ana, A. M., Albar, J. P., Martfnez-A., C. and Mellado, M. (1999). The chemokine monocyte chemoattractant protein-1 induces functional responses through dimerization of its receptor CCR2. Proc. Natl. Acad. Sci. USA 96, 3628-3633
23. Leonard, W. J. and O'Shea, J. J. (1998). JAK and STATs: Biological implications Ann, Rev. Immunol. 16, 293-322.
24. Mellado, M., Rodrfguez-Frade, J. M., Aragay, A. M., del Real, G., Vila-Coro, A., Martin de Ana, A., Serrano, A., Mayor Jr., F. and Martfnez-A., C. (1998). The chemokine MCP-1 triggers tyrosine phosphorilation of the CCR2B receptor and the JAK2/STAT3 pathway. J. Immunol. 161, 805-813.
25. Yoshimura, A., Ohkubo, T., Kiguchi, T., Jenkins, N. A., Gilbert, D. J., Copeland, N. G., Hara, T., and Miyajima, A. (1995). A novel cytokine-inducible gene CIS encodes an SH2containing protein that binds to tyrosine-phosphorylated interleukin 3 and erythropoietin receptors.EMBO J. 14, 2816-2826.
26. Starr, R., Willson, T. A., Viney, E. M., Murray, L. J., Rayner, J. R., Jenkins, B. J., Gonda, T. J., Alexander, W. S Metcalf, D., Nicola, N. A. and Hilton, D. J. (1997). A family of cytokineinducible inhibitors of signalling. Nature 387, 917-921.
27. Endo, T. A., Masuhara, M., Yokouchi, M., Suzuki, R., Sakamoto, H., Mitsui, K., Matsumoto, A., Tanimura, S., Ohtsubo, M., Misawa, H., Miyazaki, T., Leonor, N., Taniguchi, T., Fujita, T., Kanakura, Y., Komiya, S. and Yoshimura, A. (1997). A new protein containing an SH2 domain that inhibits JAK kinases. Nature 387, 921-924.
28. Naka, T., Narazaki, M., Hirata, M., Matsumoto, T., Minamoto, S., Aono, A., Nishimoto, N., Kajita, T., Taga, T., Yoshizaki, K., Akira, S. and Kishimoto, T. (1997). Structure and function of a new STAT-induced STAT inhibitor. Nature 387, 924-929.
29. Dey, B. R., Furlanetto, R. W., and Nissley, P. (2000). Suppressor of cytokine signaling (SOCS-3) protein interacts with the insulin-like growth factor-I receptor. Biochem. Biophys. Res. Commun. 278, 38-43.
30. Ogueta, S., Olazabal, I., Santos, I., Delgado-Baeza, E. and Garcia-Ruiz, J. P, (2000). Transgenic mice expressing bovine GH develop arthritic disorder and self-antibodies. J Endocrinol. 165, 321-328.
31. Gonzalo, J. A., Mazuchelli, R., Mellado, M., Frade, J. M. R., Carrera, A. C., von Kobbe, C., Merida, I. and Martfnez-A., C. (1996). Enterotoxin septic shock protection and deficient T Helper 2 cytokine production in growth hormone transgenic mice. J. Immunol. 157, 3298-3304.
32. Mellado, M., Rodriguez-Frade, J. M., Kremer, L., von Kobbe, C., Martfn de Ana, A., Merida, I. and Martfnez-A., C. (1997). Conformational changes required in the human growth hormone receptor for growth hormone signaling. J. Biol. Chem. 272, 9189-9196.
33. Ma, Q., Jones, D. and Springer, T. A. (1999). The chemokine receptor CXCR4 is required for the retention of B lineage and granulocytic precursors within the bone marrow microenvironment. Immunity 10, 463-471.
34. von Andrian, U. H. and Mackay, C. R. (2000). T cell function and migration. Advances Immunol. 343, 1020-1034.
35. Youn, B. S., Mantel, C. and Broxmeyer, H. E. (2000). Chemokines, chemokine receptors and hematopoiesis. Immunol. Rev. 177, 150-174.
36. Chen, X. P., Losman, J. A. and Rothman, P. (2000). SOCS proteins, regulators of intracellular signaling. Immunity 13, 287-290.
37. Mellado, M., Rodrfguez-Frade, J. M., Manes, S. and Martfnez-A, C. (2001). Chemokine signaling and functional responses: The role of receptor dimerization and TK pathway activation, Annual Rev. Immunol. 19, 397-421.
38. Mellado, M., Rodrfguez-Frade, J. M., Vila-Coro, A. J., Fernandez, S., Martfn de Ana, A. M., Jones, D. R., Toran, J. L. and Martfnez-A, C. (2001). Chemokine receptor homo- or heterodimerization activates distinct signaling pathways. EMBO J. (In press).
39. Hebert, T. E., Moffett, S., Morello, J-P., Loisel, T. P., Bichet, D. G., Barret, C. and Bouvier, M. 1996. A peptide derived from a p2-adrenergic receptor transmembrane domain inhibits both receptor dimerization and activation. J. Biol. Chem. 271, 28612-28616.
40. Cvejic, S. and Devi, L. A. 1997. Dimerization of the delta opioid receptor: implication for a role in receptor internalization. J. Biol. Chem. 272, 26959-26964.
41. Kuner, R., Kohr, G., Grunewald, S Eisenhardt, G., Bah, A. and Kornau, H-C. (1999). Role of heteromer formation in GABAB receptor function. Science 283, 74-77.
42. Marrero, M. B., B. Schieffer, W. G. Paxton, L. H. Eerdt, B. C. Berk, P. Delafontaine and K. E. Bernstein. (1995). Direct stimulation of Jak/STAT pathway by the angiotensin II AT receptor. Nature 375, 247-250.
43. Park, E. S., Kim, H., Suh, J. M., Park, S. J., You, S. H., Chung, H. K., Lee, K. W., Kwon, O. Y. Cho, B. Y., Kim, Y. K., Ro, H. K., Chung, J. and Shong, M. (2000) Involvement of JAK/STAT (Janus kinase/signal transducer and activator of transcription) in the thyrotropin signaling pathway. Mol. Endoc. 114, 662-670.
44. Buggy, J. J. (1998). Binding of a melanocyte-stimulating hormone to its G protein-coupled receptor on B-lymphocytes activates the JAK/STAT pathway. Biochem. J. 331, 211-216.
45. Bromberg, J. and Darnell Jr., E. (2000), The role of STATs in transcriptional control and their impact on cellular function. Oncogene 19, 2468-2473.
46. Decker, T. and Kovarik, P. (2000). Serine phosphorylation of STATs. Oncogene 19, 2628-2637.
47. Emanuelli, B., Peraldi, P., Filloux, C., Sawka-Verhelle, D., Hilton, D. and Van Obberghen, E. (2000). SOCS-3 is an insulin-induced negative regulator of insulin signaling. J. Biol. Chem. 275, 15985-15991.
48. Pezet, A., Favre, H., Kelly, P. A. and Edery, M. (1999). Inhibition and restoration of prolactin signal transduction by suppressors of cytokine signaling. J. Biol. Chem. 274, 24497-24502.
49. Sporri, B., Kovanen, P. E., Sasaki, A., Yoshimura, A. and Leonard W. J. (2001), JAB/SOCS 1/SS-1 is an interleukin-2-induced inhibitor of IL-2 signaling. Blood 97, 221-226.
50. Dorshkind, K. and Horseman, N. D. (2000) The roles of prolactin, growth hormone, insulin-like growth factor-I and thyroid hormones in lymphocyte development and function: insights from genetic models of hormone and hormone receptor deficiency. Endocr. Rev. 21, 292-312.
51. Tollet-Egnell, P., Flores-Morales, A., Stavreus-Evers, A., Sahlin, L. and Norstedt, G (1999). Growth hormone regulation of SOCS-2, SOCS-3, and CIS messenger ribonucleic acid expression in the rat. Endocrinology 140, 3693-3704.
52. Ram, P. A. and Waxman, D. J. (2000). Role of the cytokine-inducible SH2 protein CIS in desensitization of STATSb signaling by continuous growth hormone. J, Biol Chem. 275, 39487-39496.
53. Fornari, C., Palacios, F. M., Diez, R. A. and Intebi, A. D. (1994). Decreased chemotaxis of neutrophils in acromegaly and hyperprolactinemia. Eur. J. Endocrinol. 130, 463-468.
54, Perera, L. P., Goldman, C. K. and Waldmann, T. A. (1999). IL-15 induces the expression of chemokines and their receptors in T lymphocytes. J. Immunol. 162,2606-2612.

Incorporation by reference. All patent documents and publications cited in this application are incorporated by reference in their entirety for all purposes to the same extent as if each individual publication or patent document were so individually denoted. More particularly, but not exclusively, the publication Mellado et al., Chemokine Receptor Homo- or Heterodimerization Activates Distinct Signaling Pathways, 20 EMBO J. 2497 (2001) is specifically incorporated by reference in its entirety for all purposes.

1. A method of identifying compounds for use in a pharmaceutical composition having an anti-viral effect against CXCR4-dependent HIV activity comprising:
providing a first aliquot of CXCR4-expressing GH-Tg cells;
contacting said first aliquot with HIV particles;
providing a second aliquot of CXC R4-expressing GH-Tg cells;
contacting said second aliquot with a test ligand;
contacting said second aliquot with HIV particles; and
isolating virus from said first and said second aliquot of cells, wherein a decrease in the ability to isolate virus from said second aliquot of cells indicates said test ligand possess anti-viral activity against HIV.

2. The method of identifying compounds for use in a pharmaceutical composition having an anti-viral effect against CXCR4-dependent HIV activity, according to claim 1, wherein said second aliquot of cells is contacted with a plurality of test ligands.

3. A method of identifying compounds for use in a pharmaceutical composition having a therapeutic effect against a disease involving CXCR4-dependent chemotaxis comprising
providing a first aliquot of CXCR4-expressing GH-Tg cells;
contacting said first aliquot with CXCL12;
measuring a first migration index;
providing a second aliquot of CXCR4-expressing GH-Tg cells;
contacting said second aliquot with CXCL12;
contacting said second aliquot with a test ligand;
measuring a second migration index; and
determining a therapeutic potential.

4. The method of identifying compounds for use in a pharmaceutical composition having a therapeutic effect against a disease involving CXCR4-dependent chemotaxis, according to claim 3, wherein
said disease is associated with aberrant leukocyte recruitment or activation, said disease being selected from the group consisting of arthritis, psoriasis, multiple sclerosis, ulcerative colitis, Crohn's disease, allergy, asthma, AIDS associated encephalitis, AIDS related maculopapular skin eruption, AIDS related interstitial pneumonia, AIDS related enteropathy, AIDS related periportal hepatic inflammation and AIDS related glomerulo nephritis

5. The method of identifying compounds for use in a pharmaceutical composition having a therapeutic effect against a disease involving CXCR4-dependent chemotaxis, according to claim 3, wherein
said cell is a CXCR4 expressing cell selected from the group consisting of thymocytes, CD34+ cells, B lymphocytes, T lymphocytes, dendritic cells, macrophages, neutrophils, and platelets.

6. The method of identifying compounds for use in a pharmaceutical composition having a therapeutic effect against a disease involving CXCR4-dependent chemotaxis, according to claim 5, wherein said cell is an IM-9 cell.

7. A method of identifying compounds for use in a pharmaceutical composition having a therapeutic effect against a SOCS3 inhibitable disease comprising:
providing a first cell having at least one chemokine receptor expressed thereon, said first cell having been transfected with at least one SOCS construct;
contacting said first cell with at least one chemokine;
measuring a first migration index;
providing a second cell having at least one chemokine receptor expressed thereon, said second cell having been transfected with at least one SOCS construct;
contacting said second cell with at least one chemokine;
measuring a second migration index; and
determining a therapeutic potential.

8. The method of identifying compounds for use in a pharmaceutical composition having a therapeutic effect against a SOCS3 inhibitable disease, according to claim 7, wherein said cells are HEK-293 cells.

9. The method of identifying compounds for use in a pharmaceutical composition having a therapeutic effect against a SOCS3 inhibitable disease, according to claim 7, wherein said cells are IM-9 cells.

10. A method of treating a subject having a disease associated with CXCR4-dependent HIV comprising administering to said subject a therapeutically anti-viral effective amount of a compound that induces the expression of SOCS3 and a pharmaceutically acceptable carrier.

11. The method of treating a subject having a disease associated with CXCR4-dependent HIV, according to claim 10, wherein said subject is an animal in need of such treatment selected from the group cossisting of humans, domestic animals, and laboratory animals.

12. The method of treating a subject having a disease associated with CXCR4-dependent HIV, according to claim 10, wherein said molecule binds to GHR.


SEQUENCE LISTING (2 sequences)

SEQ ID NO: 1 - DNA, 27 bp, Artificial
  wild type; wt SIE
ggggtgcatttcccgtaaatcttgtct

SEQ ID NO: 2 - DNA, 27 bp, Artificial
  mut SIE
ggggtgcatccaccgtaaatcttgtct